(19) United States
(12) Patent Application Publication
(10) Pub. No.: US 2013/0244225 A1
(43) Pub. Date: Sep. 19, 2013

(54) MICROORGANISM CONCENTRATION PROCESS AND DEVICE

(71) Applicants: Manjiri T. Kshirsagar, Woodbury, MN (US); Andrew W. Rabins, St. Paul, MN (US)

(72) Inventors: Manjiri T. Kshirsagar, Woodbury, MN (US); Andrew W. Rabins, St. Paul, MN (US)

(21) Appl. No.: 13/825,676

(22) PCT Filed: Nov. 23, 2011
(86) PCT No.: PCT/US11/62069

§ 371 Date: Mar. 22, 2013

Related U.S. Application Data
(60) Provisional application No. 61/420,045, filed on Dec. 6, 2010.

(51) Int. Cl. G01N 1/40; G01N 33/569

(52) U.S. Cl. CPC G01N 1/405; G01N 33/56911
USPC 435/5; 435/7.92; 435/309.1; 427/212

(57) ABSTRACT

A process for capturing or concentrating microorganisms for detection or assay comprises (a) providing a concentration device comprising (1) a porous fibrous nonwoven matrix and (2) a plurality of particles of at least one concentration agent that comprises diatomaceous earth, the particles being enmeshed in the porous fibrous nonwoven matrix; (b) providing a sample comprising at least one target cellular analyte; (c) contacting the concentration device with the sample such that at least a portion of the at least one target cellular analyte is bound to or captured by the concentration device; and (d) detecting the presence of at least one bound target cellular analyte.

### FIELD

This invention relates to processes for capturing or concentrating microorganisms such that they remain viable for detection or assay. In other aspects, this invention also relates to concentration devices (and diagnostic kits comprising the devices) for use in carrying out such processes and to methods for device preparation.

### BACKGROUND

Food-borne illnesses and hospital-acquired infections resulting from microorganism contamination are a concern in numerous locations all over the world. Thus, it is often desirable or necessary to assay for the presence of bacteria or other microorganisms in various clinical, food, environmental, or other samples, in order to determine the identity and/or the quantity of the microorganisms present.

Bacterial DNA or bacterial RNA, for example, can be assayed to assess the presence or absence of a particular bacterial species even in the presence of other bacterial species. The ability to detect the presence of a particular bacterium, however, depends, at least in part, on the concentration of the bacterium in the sample being analyzed. Bacterial samples can be plated or cultured to increase the numbers of the bacteria in the sample to ensure an adequate level for detection, but the culturing step often requires substantial time and therefore can significantly delay the assessment results.

Concentration of the bacteria in the sample can shorten the culturing time or even eliminate the need for a culturing step. Thus, methods have been developed to isolate (and thereby concentrate) particular bacterial strains by using antibodies specific to the strain (for example, in the form of antibody-coated magnetic or non-magnetic particles). Such methods, however, have tended to be expensive and still somewhat slower than desired for at least some diagnostic applications.

Concentration methods that are not strain-specific have also been used (for example, to obtain a more general assessment of the microorganisms present in a sample). After concentration of a mixed population of microorganisms, the presence of particular strains can be determined, if desired, by using strain-specific probes.

Non-specific concentration or capture of microorganisms has been achieved through methods based upon carbohydrate and lectin protein interactions. Chitosan-coated supports have been used as non-specific capture devices, and substances (for example, carbohydrates, vitamins, iron-chelating compounds, and siderophores) that serve as nutrients for microorganisms have also been described as being useful as ligands to provide non-specific capture of microorganisms.

Various inorganic materials (for example, hydroxyapatite and metal hydroxides) have been used to non-specifically bind and concentrate bacteria. Physical concentration methods (for example, filtration, chromatography, centrifugation, and gravitational settling) have also been utilized for non-specific capture, with and/or without the use of inorganic binding agents. Such non-specific concentration methods have varied in speed (at least some food testing procedures still requiring at least overnight incubation as a primary cultural enrichment step), cost (at least some requiring expensive equipment, materials, and/or trained technicians), sample requirements (for example, sample nature and/or volume limitations), space requirements, ease of use (at least some requiring complicated multi-step processes), suitability for on-site use, and/or effectiveness.

### SUMMARY

Thus, we recognize that there is an urgent need for processes for rapidly detecting pathogenic microorganisms. Such processes will preferably be not only rapid but also low in cost, simple (involving no complex equipment or procedures), and/or effective under a variety of conditions (for example, with varying types of sample matrices and/or pathogenic microorganisms, varying microorganism loads, and varying sample volumes).

Briefly, in one aspect, this invention provides a process for non-specifically concentrating the strains of microorganisms (for example, strains of bacteria, fungi, yeasts, protozoans, viruses (including both non-enveloped and enveloped viruses), and bacterial endospores) present in a sample, such that the microorganisms remain viable for the purpose of detection or assay of one or more of the strains. The process comprises (a) providing a concentration device comprising (1) a porous fibrous nonwoven matrix and (2) a plurality of particles of at least one concentration agent that comprises diatomaceous earth (preferably, surface-modified diatomaceous earth), the particles being enmeshed in the porous fibrous nonwoven matrix; (b) providing a sample (preferably, in the form of a fluid) comprising at least one target cellular analyte (for example, at least one microorganism strain); (c) contacting the concentration device with the sample (preferably, by passing the sample through the concentration device) such that at least a portion of the at least one target cellular analyte is bound to or captured by the concentration device; and (d) detecting the presence of at least one bound target cellular analyte.

The process can optionally further comprise separating the concentration device from the sample and/or culturally enriching at least one bound target cellular analyte (for example, by incubating the separated concentration device in a general or microorganism-specific culture medium, depending upon whether general or selective microorganism enrichment is desired) and/or isolating or separating captured target cellular analytes (for example, microorganisms or one or more components thereof) from the concentration device after sample contacting (for example, by passing an elution agent or a lysis agent through the concentration device). If desired, however, detection of the target cellular analyte (for example, by culture-based, microscopy/imaging, genetic, luminescence-based, or immunologic detection methods) generally can be carried out in the presence of the concentration device.

The process of the invention does not target a specific cellular analyte (for example, a particular microorganism strain). Rather, it has been discovered that a concentration device comprising certain relatively inexpensive, inorganic materials enmeshed in a porous fibrous nonwoven matrix can be surprisingly effective in capturing a variety of microorganisms (and surprisingly effective in isolating or separating the captured microorganisms via elution, relative to corresponding devices without the inorganic material). Such devices can be used to concentrate the microorganism strains present in a sample (for example, a food sample) in a non-strain-specific manner, so that one or more of the microorganism strains (preferably, one or more strains of bacteria) can be more easily and rapidly assayed.

The process of the invention is relatively simple and low in cost (requiring no complex equipment or expensive strain-specific materials) and can be relatively fast (preferred embodiments capturing at least about 70 percent (more preferably, at least about 80 percent; most preferably, at least about 90 percent) of the microorganisms present in a relatively homogeneous fluid sample in less than about 10 minutes, relative to a corresponding control sample having no contact with the concentration device). In contrast with the use of particulate concentration agents alone, the process can be surprisingly effective in microorganism capture with only relatively short sample contact times (for example, as short as about 20 seconds) and without the need for a settling step.

The process of the invention is also surprisingly “assay-friendly.” Detection can generally be effected in the presence of the concentration device without significant assay interference (for example, without detection errors resulting from the absorption of assay reagents by the concentration device or resulting from the leaching of assay inhibitors from the concentration device). This enables concentration and detection to be carried out quickly (for example, as quickly as 10 minutes or less) in the sampling environment.

In addition, the process can be effective with a variety of microorganisms (including pathogens such as both gram positive and gram negative bacteria) and with a variety of samples (different sample matrices and, unlike at least some prior art methods, even samples having low microorganism content and/or large volumes). Thus, at least some embodiments of the process of the invention can meet the above-cited urgent need for low-cost, simple processes for rapidly detecting pathogenic microorganisms under a variety of conditions.

The process of the invention can be especially advantageous for concentrating the microorganisms in food samples (for example, particulate-containing food samples, especially those comprising relatively coarse particulates), as the concentration device used in the process can exhibit at least somewhat greater resistance to clogging than at least some filtration devices such as absolute micron filters. This can facilitate more complete sample processing (which is essential to eliminating false negative assays in food testing) and the handling of relatively large volume samples (for example, under field conditions).

A preferred concentration process comprises
(a) providing a concentration device comprising
   (1) a porous fibrous nonwoven matrix comprising (i) at least one fibrillated fiber and (ii) at least one polymeric binder, and
   (2) a plurality of particles of at least one concentration agent that comprises diatomaceous earth bearing, on at least a portion of its surface, a surface treatment comprising a surface modifier comprising metal oxide (preferably, titanium dioxide or ferric oxide), fine-nanoscale gold or platinum, or a combination thereof; the particles being enmeshed in the porous fibrous nonwoven matrix;
(b) providing a fluid sample comprising at least one target cellular analyte; and
(c) passing the fluid sample through the concentration device in a manner such that at least a portion of the at least one target cellular analyte is bound to or captured by the concentration device.

In another aspect, the invention also provides a concentration device comprising (a) a porous fibrous nonwoven matrix; and (b) a plurality of particles of at least one concentration agent that comprises diatomaceous earth bearing, on at least a portion of its surface, a surface treatment comprising a surface modifier comprising metal oxide (preferably, titanium dioxide or ferric oxide), fine-nanoscale gold or platinum, or a combination thereof; wherein the particles are enmeshed in the porous fibrous nonwoven matrix. The invention also provides a diagnostic kit for use in carrying out the concentration process of the invention, the kit comprising (a) at least one concentration device of the invention; and (b) at least one testing container or testing reagent for use in carrying out the above-described concentration process.

In yet another aspect, the invention provides a process for preparing a concentration device comprising (a) providing a plurality of fibers; (b) providing a plurality of particles of at least one concentration agent that comprises diatomaceous earth bearing, on at least a portion of its surface, a surface treatment comprising a surface modifier comprising metal oxide (preferably, titanium dioxide or ferric oxide), fine-nanoscale gold or platinum, or a combination thereof; and (c) forming at least a portion of the plurality of fibers into a porous fibrous nonwoven matrix having at least a portion of the plurality of particles enmeshed therein.

In still another aspect, the invention also provides a filter media comprising (a) a porous fibrous nonwoven matrix; and (b) a plurality of particles of at least one concentration agent that comprises diatomaceous earth bearing, on at least a portion of its surface, a surface treatment comprising a surface modifier comprising metal oxide, fine-nanoscale gold or platinum, or a combination thereof; wherein the particles are enmeshed in the porous fibrous nonwoven matrix.

### DETAILED DESCRIPTION

In the following detailed description, various sets of numerical ranges (for example, of the number of carbon atoms in a particular moiety, of the amount of a particular component, or the like) are described, and, within each set, any lower limit of a range can be paired with any upper limit of a range. Such numerical ranges also are meant to include all numbers subsumed within the range (for example, 1 to 5 includes 1, 1.5, 2, 2.75, 3, 3.80, 4, 5, and so forth).

As used herein, the term “and/or” means one or all of the listed elements or a combination of any two or more of the listed elements.

The words “preferred” and “preferably” refer to embodiments of the invention that may afford certain benefits under certain circumstances. Other embodiments may also be preferred, however, under the same or other circumstances. Furthermore, the recitation of one or more preferred embodiments does not imply that other embodiments are not useful, and is not intended to exclude other embodiments from the scope of the invention.

The term “comprises” and variations thereof do not have a limiting meaning where these terms appear in the description and claims.

As used herein, “a,” “an,” “the,” “at least one,” and “one or more” are used interchangeably.

The above “Summary of the Invention” section is not intended to describe every embodiment or every implementation of the invention. The detailed description that follows more particularly describes illustrative embodiments. Throughout the detailed description, guidance is provided through lists of examples, which examples can be used in various combinations. In each instance, a recited list serves only as a representative group and should not be interpreted as being an exclusive list.

### DEFINITIONS

As used in this patent application:

“aramid” means an aromatic polyamide;
“cellular analyte” means an analyte of cellular origin (that is, a microorganism or a component thereof (for example, a cell or a cellular component such as deoxyribonucleic acid (DNA) or ribonucleic acid (RNA), proteins, nucleotides such as adenosine triphosphate (ATP), and the like, and combinations thereof); references to a microorganism or microorganism strain throughout this specification are meant to apply more generally to any cellular analyte);
“concentration agent” means a material or composition that binds cellular analytes (preferably, having a cellular analyte capture or binding efficiency of at least about 60 percent; more preferably, at least about 70 percent; even more preferably, at least about 80 percent; most preferably, at least about 90 percent);
“culture device” means a device that can be used to propagate microorganisms under conditions that will permit at least one cell division to occur (preferably, culture devices include a housing to reduce or minimize the possibility of incidental contamination and/or a source of nutrients to support the growth of microorganisms);
“detection” means the identification of a cellular analyte (for example, at least a component of a target microorganism, which thereby determines that the target microorganism is present);
“enmeshed” (in regard to particles of concentration agent in a fibrous nonwoven matrix) means that the particles are entrapped in the fibrous nonwoven matrix (and, preferably, distributed within it), rather than merely being borne on its surface;
“fibrillated” (in regard to fibers or fibrous material) means treated (for example, by beating) in a manner that forms fibrils or branches attached to a fiber's main trunk;
“fibrous nonwoven matrix” means a web or medium, other than a woven or knitted fabric, comprising interlaid fibers (for example, a web comprising fibers that are interlaid by meltblowing, spunbonding, or other air laying techniques; carding; wet laying; or the like);
“genetic detection” means the identification of a component of genetic material such as DNA or RNA that is derived from a target microorganism;
“immunologic detection” means the identification of an antigenic material such as a protein or a proteoglycan that is derived from a target microorganism;
“microorganism” means any cell or particle having genetic material suitable for analysis or detection (including, for example, bacteria, yeasts, viruses, and bacterial endospores);
“microorganism strain” means a particular type of microorganism that is distinguishable through a detection method (for example, microorganisms of different genera, of different species within a genera, or of different isolates within a species);
“para-aramid” means an aromatic polyamide having its amide linkages bonded to substituted (for example, alkyl-substituted) or unsubstituted benzene rings in para-relation (bonded to carbon numbers one and four);
“sample” means a substance or material that is collected (for example, to be analyzed);
“sample matrix” means the components of a sample other than cellular analytes;
“target cellular analyte” means any cellular analyte that is desired to be detected;
“target microorganism” means any microorganism that is desired to be detected; and
“through pore” (in reference to a porous matrix) means a pore that comprises a passageway or channel (with separate inlet and outlet) through the matrix.

### Concentration Agent

Concentration agents suitable for use in carrying out the process of the invention include those particulate concentration agents that comprise diatomaceous earth. The diatomaceous earth can be used in natural (untreated) form or can be surface-modified (for example, by deposition of another material or by other known or hereafter-developed surface treatment methods) to enhance its ability to concentrate microorganisms (preferably, the diatomaceous earth is surface-modified).

Concentration or capture using such concentration agents is generally not specific to any particular strain, species, or type of microorganism and therefore provides for the concentration of a general population of microorganisms in a sample. Specific strains of microorganisms can then be detected from among the captured microorganism population using any known detection method with strain-specific probes. Thus, the concentration agents can be used for the detection of microbial contaminants or pathogens (particularly food-borne pathogens such as bacteria) in clinical, food, environmental, or other samples.

When dispersed or suspended in water systems, inorganic materials such as diatomaceous earth exhibit surface charges that are characteristic of the material and the pH of the water system. The potential across the material-water interface is called the “zeta potential,” which can be calculated from electrophoretic mobilities (that is, from the rates at which the particles of material travel between charged electrodes placed in the water system). Preferably, the concentration agents have a negative zeta potential at a pH of about 7.

In carrying out the process of the invention, the concentration agents can be used in essentially any particulate form (preferably, a relatively dry or volatiles-free form) that is amenable to blending with fibers to form the concentration device used in the process. For example, the concentration agents can be used in powder form or can be applied to a particulate support such as beads or the like.

Preferably, the concentration agents are used in the form of a powder. Useful powders include those that comprise microparticles (preferably, microparticles having a particle size in the range of about 1 micrometer (more preferably, about 2 micrometers; even more preferably, about 3 micrometers; most preferably, about 4 micrometers) to about 100 micrometers (more preferably, about 50 micrometers; even more preferably, about 25 micrometers; most preferably, about 15 or 20 micrometers; where any lower limit can be paired with any upper limit of the range, as referenced above).

Surface-modified diatomaceous earth concentration agents suitable for use in carrying out the process of the invention include those that comprise diatomaceous earth bearing, on at least a portion of its surface, a surface treatment comprising a surface modifier comprising metal oxide (preferably, titanium dioxide or ferric oxide), fine-nanoscale gold or platinum, or a combination thereof (preferably, a surface modifier comprising at least one metal oxide). Such concentration agents include those described in U.S. Patent Application Publication No. US 2010/0209961 published on Aug. 19, 2010 (Kshirsagar et al.; 3M Innovative Properties Company), the descriptions of the concentration agents and methods of their preparation being incorporated herein by reference.

The surface treatment preferably further comprises a metal oxide selected from ferric oxide, zinc oxide, aluminum oxide, and the like, and combinations thereof (more preferably, ferric oxide). Although noble metals such as gold have been known to exhibit antimicrobial characteristics, the gold-containing concentration agents used in the process of the invention surprisingly can be effective not only in concentrating the microorganisms but also in leaving them viable for purposes of detection or assay.

Useful surface modifiers include fine-nanoscale gold; fine-nanoscale platinum; fine-nanoscale gold in combination with at least one metal oxide (preferably, titanium dioxide, ferric oxide, or a combination thereof); titanium dioxide; titanium dioxide in combination with at least one other (that is, other than titanium dioxide) metal oxide; ferric oxide; ferric oxide in combination with at least one other (that is, other than ferric oxide) metal oxide; and the like; and combinations thereof. Preferred surface modifiers include fine-nanoscale gold; fine-nanoscale platinum; fine-nanoscale gold in combination with at least ferric oxide or titanium dioxide; titanium dioxide; ferric oxide; titanium dioxide in combination with at least ferric oxide; and combinations thereof.

More preferred surface modifiers include fine-nanoscale gold; fine-nanoscale platinum; fine-nanoscale gold in combination with ferric oxide or titanium dioxide; titanium dioxide; titanium dioxide in combination with ferric oxide; ferric oxide; and combinations thereof (even more preferably, fine-nanoscale gold; fine-nanoscale gold in combination with ferric oxide or titanium dioxide; titanium dioxide in combination with ferric oxide; titanium dioxide; ferric oxide; and combinations thereof). Ferric oxide, titanium dioxide, and combinations thereof are most preferred.

At least some of the surface-modified diatomaceous earth concentration agents have zeta potentials that are at least somewhat more positive than that of untreated diatomaceous earth, and the concentration agents can be surprisingly significantly more effective than untreated diatomaceous earth in concentrating microorganisms such as bacteria, the surfaces of which generally tend to be negatively charged. Preferably, the concentration agents have a negative zeta potential at a pH of about 7 (more preferably, a zeta potential in the range of about −5 millivolts to about −20 millivolts at a pH of about 7; even more preferably, a zeta potential in the range of about −8 millivolts to about −19 millivolts at a pH of about 7; most preferably, a zeta potential in the range of about −10 millivolts to about −18 millivolts at a pH of about 7).

The surface-modified diatomaceous earth concentration agents comprising fine-nanoscale gold or platinum can be prepared by depositing gold or platinum on diatomaceous earth by physical vapor deposition (optionally, by physical vapor deposition in an oxidizing atmosphere). As used herein, the term “fine-nanoscale gold or platinum” refers to gold or platinum bodies (for example, particles or atom clusters) having all dimensions less than or equal to 5 nanometers (nm) in size. Preferably, at least a portion of the deposited gold or platinum has all dimensions (for example, particle diameter or atom cluster diameter) in the range of up to (less than or equal to) about 10 nm in average size (more preferably, up to about 5 nm; even more preferably, up to about 3 nm).

In most preferred embodiments, at least a portion of the gold is ultra-nanoscale (that is, having at least two dimensions less than 0.5 nm in size and all dimensions less than 1.5 nm in size). The size of individual gold or platinum nanoparticles can be determined by transmission electron microscopy (TEM) analysis, as is well known in the art.

Diatomaceous earth (or kieselguhr) is a natural siliceous material produced from the remnants of diatoms, a class of ocean-dwelling microorganisms. Thus, it can be obtained from natural sources and is also commercially available (for example, from Alfa Aesar, A Johnson Matthey Company, Ward Hill, Mass.). Diatomaceous earth particles generally comprise small, open networks of silica in the form of symmetrical cubes, cylinders, spheres, plates, rectangular boxes, and the like. The pore structures in these particles can generally be remarkably uniform.

Diatomaceous earth can be used as the raw, mined material or as purified and optionally milled particles. Preferably, the diatomaceous earth is in the form of milled particles with sizes in the range of about 1 micrometer to about 50 micrometers in diameter (more preferably, about 3 micrometers to about 10 micrometers).

The diatomaceous earth can optionally be heat treated prior to use to remove any vestiges of organic residues. If a heat treatment is used, it can be preferable that the heat treatment be at 500° C. or lower, as higher temperatures can produce undesirably high levels of crystalline silica.

The amount of gold or platinum provided on the diatomaceous earth can vary over a wide range. Since gold and platinum are expensive, it is desirable not to use more than is reasonably needed to achieve a desired degree of concentration activity. Additionally, because nanoscale gold or platinum can be highly mobile when deposited using PVD, activity can be compromised if too much gold or platinum is used, due to coalescence of at least some of the gold or platinum into large bodies.

For these reasons, the weight loading of gold or platinum on the diatomaceous earth preferably is in the range of about 0.005 (more preferably, 0.05) to about 10 weight percent, more preferably about 0.005 (even more preferably, 0.05) to about 5 weight percent, and even more preferably from about 0.005 (most preferably, 0.05) to about 2.5 weight percent, based upon the total weight of the diatomaceous earth and the gold or platinum.

Gold and platinum can be deposited by PVD techniques (for example, by sputtering) to form concentration-active, fine-nanoscale particles or atom clusters on a support surface. It is believed that the metal is deposited mainly in elemental form, although other oxidation states may be present.

In addition to gold and/or platinum, one or more other metals can also be provided on the same diatomaceous earth supports and/or on other supports intermixed with the gold- and/or platinum-containing supports. Examples of such other metals include silver, palladium, rhodium, ruthenium, osmium, copper, iridium, and the like, and combinations thereof. If used, these other metals can be co-deposited on a support from a target source that is the same or different from the gold or platinum source target that is used. Alternatively, such metals can be provided on a support either before or after the gold and/or platinum is deposited. Metals requiring a thermal treatment for activation advantageously can be applied to a support and heat treated before the gold and/or platinum is deposited.

Physical vapor deposition refers to the physical transfer of metal from a metal-containing source or target to a support medium. Physical vapor deposition can be carried out in various different ways. Representative approaches include sputter deposition (preferred), evaporation, and cathodic arc deposition. Any of these or other PVD approaches can be used in preparing the concentration agents used in carrying out the process of the invention, although the nature of the PVD technique can impact the resulting activity. PVD can be carried out by using any of the types of apparatus that are now used or hereafter developed for this purpose.

Physical vapor deposition preferably is performed while the support medium to be treated is being well-mixed (for example, tumbled, fluidized, milled, or the like) to ensure adequate treatment of support surfaces. Methods of tumbling particles for deposition by PVD are described in U.S. Pat. No. 4,618,525 (Chamberlain et al.), the description of which is incorporated herein by reference. When carrying out PVD on fine particles or fine particle agglomerates (for example, less than about 10 micrometers in average diameter), the support medium is preferably both mixed and comminuted (for example, ground or milled to some degree) during at least a portion of the PVD process.

Physical vapor deposition can be carried out at essentially any desired temperature(s) over a very wide range. However, the deposited metal can be more active (perhaps due to more defects and/or lower mobility and coalescence) if the metal is deposited at relatively low temperatures (for example, at a temperature below about 150° C., preferably below about 50° C., more preferably at ambient temperature (for example, about 20° C. to about 27° C.) or less). Operating under ambient conditions can be generally preferred as being effective and economical, as no heating or chilling is required during the deposition.

The physical vapor deposition can be carried out in an inert sputtering gas atmosphere (for example, in argon, helium, xenon, radon, or a mixture of two or more thereof (preferably, argon)), and, optionally, the physical vapor deposition can be carried out in an oxidizing atmosphere. The oxidizing atmosphere preferably comprises at least one oxygen-containing gas (more preferably, an oxygen-containing gas selected from oxygen, water, hydrogen peroxide, ozone, and combinations thereof; even more preferably, an oxygen-containing gas selected from oxygen, water, and combinations thereof; most preferably, oxygen). The oxidizing atmosphere further comprises an inert sputtering gas such as argon, helium, xenon, radon, or a mixture of two or more thereof (preferably, argon). The total gas pressure (all gases) in the vacuum chamber during the PVD process can be from about 1 mTorr to about 25 mTorr (preferably, from about 5 mTorr to about 15 mTorr). The oxidizing atmosphere can comprise from about 0.05 percent to about 60 percent by weight oxygen-containing gas (preferably, from about 0.1 percent to about 50 percent by weight; more preferably, from about 0.5 percent to about 25 percent by weight), based upon the total weight of all gases in the vacuum chamber.

The diatomaceous earth support medium can optionally be calcined prior to metal deposition, although this can increase its crystalline silica content. Since gold and platinum are active right away when deposited via PVD, there is generally no need for heat treatment after metal deposition, unlike deposition by some other methodologies. Such heat treating or calcining can be carried out if desired, however, to enhance activity.

In general, thermal treatment can involve heating the support at a temperature in the range of about 125° C. to about 1000° C. for a time period in the range of about 1 second to about 40 hours, preferably about 1 minute to about 6 hours, in any suitable atmosphere such as air, an inert atmosphere such as nitrogen, carbon dioxide, argon, a reducing atmosphere such as hydrogen, and the like. The particular thermal conditions to be used can depend upon various factors including the nature of the support.

Generally, thermal treatment can be carried out below a temperature at which the constituents of the support would be decomposed, degraded, or otherwise unduly thermally damaged. Depending upon factors such as the nature of the support, the amount of metal, and the like, activity can be compromised to some degree if the system is thermally treated at too high a temperature.

The surface-modified diatomaceous earth concentration agents comprising metal oxide can be prepared by depositing metal oxide on diatomaceous earth by hydrolysis of a hydrolyzable metal oxide precursor compound. Suitable metal oxide precursor compounds include metal complexes and metal salts that can be hydrolyzed to form metal oxides. Useful metal complexes include those comprising alkoxide ligands, hydrogen peroxide as a ligand, carboxylate-functional ligands, and the like, and combinations thereof. Useful metal salts include metal sulfates, nitrates, halides, carbonates, oxalates, hydroxides, and the like, and combinations thereof.

When using metal salts or metal complexes of hydrogen peroxide or carboxylate-functional ligands, hydrolysis can be induced by either chemical or thermal means. In chemically-induced hydrolysis, the metal salt can be introduced in the form of a solution into a dispersion of the diatomaceous earth, and the pH of the resulting combination can be raised by the addition of a base solution until the metal salt precipitates as a hydroxide complex of the metal on the diatomaceous earth. Suitable bases include alkali metal and alkaline earth metal hydroxides and carbonates, ammonium and alkyl-ammonium hydroxides and carbonates, and the like, and combinations thereof. The metal salt solution and the base solution can generally be about 0.1 to about 2 M in concentration.

Preferably, the addition of the metal salt to the diatomaceous earth is carried out with stirring (preferably, rapid stirring) of the diatomaceous earth dispersion. The metal salt solution and the base solution can be introduced to the diatomaceous earth dispersion separately (in either order) or simultaneously, so as to effect a preferably substantially uniform reaction of the resulting metal hydroxide complex with the surface of the diatomaceous earth. The reaction mixture can optionally be heated during the reaction to accelerate the speed of the reaction. In general, the amount of base added can equal the number of moles of the metal times the number of non-oxo and non-hydroxo counterions on the metal salt or metal complex.

Alternatively, when using salts of titanium or iron, the metal salt can be thermally induced to hydrolyze to form the hydroxide complex of the metal and to interact with the surface of the diatomaceous earth. In this case, the metal salt solution can generally be added to a dispersion of the diatomaceous earth (preferably, a stirred dispersion) that has been heated to a sufficiently high temperature (for example, greater than about 50° C.) to promote the hydrolysis of the metal salt. Preferably, the temperature is between about 75° C. and 100° C., although higher temperatures can be used if the reaction is carried out in an autoclave apparatus.

When using metal alkoxide complexes, the metal complex can be induced to hydrolyze to form a hydroxide complex of the metal by partial hydrolysis of the metal alkoxide in an alcohol solution. Hydrolysis of the metal alkoxide solution in the presence of diatomaceous earth can result in metal hydroxide species being deposited on the surface of the diatomaceous earth.

Alternatively, the metal alkoxide can be hydrolyzed and deposited onto the surface of the diatomaceous earth by reacting the metal alkoxide in the gas phase with water, in the presence of the diatomaceous earth. In this case, the diatomaceous earth can be agitated during the deposition in either, for example, a fluidized bed reactor or a rotating drum reactor.

After the above-described hydrolysis of the metal oxide precursor compound in the presence of the diatomaceous earth, the resulting surface-treated diatomaceous earth can be separated by settling or by filtration or by other known techniques. The separated product can be purified by washing with water and can then be dried (for example, at 50° C. to 150° C.).

Although the surface-treated diatomaceous earth generally can be functional after drying, it can optionally be calcined to remove volatile by-products by heating in air to about 250° C. to 650° C. generally without loss of function. This calcining step can be preferred when metal alkoxides are utilized as the metal oxide precursor compounds.

In general, with metal oxide precursor compounds of iron, the resulting surface treatments comprise nanoparticulate iron oxide. When the weight ratio of iron oxide to diatomaceous earth is about 0.08, X-ray diffraction (XRD) does not show the presence of a well-defined iron oxide material. Rather, additional X-ray reflections are observed at 3.80, 3.68, and 2.94 Å. TEM examination of this material shows the surface of the diatomaceous earth to be relatively uniformly coated with globular nanoparticulate iron oxide material. The crystallite size of the iron oxide material is less than about 20 nm, with most of the crystals being less than about 10 nm in diameter. The packing of these globular crystals on the surface of the diatomaceous earth is dense in appearance, and the surface of the diatomaceous earth appears to be roughened by the presence of these crystals.

In general, with metal oxide precursor compounds of titanium, the resulting surface treatments comprise nanoparticulate titania. When depositing titanium dioxide onto diatomaceous earth, XRD of the resulting product after calcination to about 350° C. can show the presence of small crystals of anatase titania. With relatively lower titanium/diatomaceous earth ratios or in cases where mixtures of titanium and iron oxide precursors are used, no evidence of anatase is generally observed by X-ray analysis.

Since titania is well-known as a potent photo-oxidation catalyst, the titania-modified diatomaceous earth concentration agents can be used to concentrate microorganisms for analysis and then optionally also be used as photoactivatable agents for killing residual microorganisms and removing unwanted organic impurities after use. Thus, the titania-modified diatomaceous earth can both isolate biomaterials for analysis and then be photochemically cleaned for re-use. These materials can also be used in filtration applications where microorganism removal as well as antimicrobial effects can be desired.

Other particularly preferred concentration agents suitable for use in carrying out the process of the invention include those that comprise an adsorption buffer-modified, surface-modified diatomaceous earth. Such concentration agents include those described in U.S. Provisional Patent Application No. 61/289,213 filed on Dec. 22, 2009 (Kshirsagar; 3M Innovative Properties Company), the descriptions of the concentration agents and methods of their preparation being incorporated herein by reference.

### Concentration Device

Concentration devices suitable for use in carrying out the process of the invention include those that comprise (a) a porous fibrous nonwoven matrix and (b) a plurality of the above-described concentration agent particles, the particles being enmeshed in the porous fibrous nonwoven matrix. Such concentration devices can be prepared by essentially any process that is capable of providing a fibrous nonwoven matrix (that is, a web or medium, other than a woven or knitted fabric, comprising interlaid fibers) having the concentration agent particles enmeshed therein. Useful processes include meltblowing, spunbonding, and other air laying techniques; carding; wet laying; and the like; and combinations thereof (preferably, air laying, wet laying, and combinations thereof; more preferably, wet laying).

Fibers that are suitable for use in preparing the porous fibrous nonwoven matrix of the concentration device include pulpable fibers. Preferred pulpable fibers are those that are stable to radiation and/or to a variety of solvents. Useful fibers include polymeric fibers, inorganic fibers, and combinations thereof (preferably, polymeric fibers and combinations thereof). Preferably, at least some of the fibers that are utilized exhibit a degree of hydrophilicity.

Suitable polymeric fibers include those made from natural (animal or vegetable) and/or synthetic polymers, including thermoplastic and solvent-dispersible polymers. Useful polymers include wool; silk; cellulosic polymers (for example, cellulose, cellulose derivatives, and the like); fluorinated polymers (for example, poly(vinyl fluoride), poly(vinylidene fluoride), copolymers of vinylidene fluoride such as poly(vinylidene fluoride-co-hexafluoropropylene), copolymers of chlorotrifluoroethylene such as poly(ethylene-co-chlorotrifluoroethylene), and the like); chlorinated polymers; polyolefins (for example, poly(ethylene), poly(propylene), poly(1-butene), copolymers of ethylene and propylene, alpha olefin copolymers such as copolymers of ethylene or propylene with 1-butene, 1-hexene, 1-octene, and 1-decene, poly(ethylene-co-1-butene), poly(ethylene-co-1-butene-co-1-hexene), and the like); poly(isoprenes); poly(butadienes); polyamides (for example, nylon 6; nylon 6,6; nylon 6,12; poly(iminoadipoyliminohexamethylene); poly(iminoadipoyliminodecamethylene); polycaprolactam; and the like); polyimides (for example, poly(pyromellitimide) and the like); polyethers; poly(ether sulfones) (for example, poly(diphenylether sulfone), poly(diphenylsulfone-co-diphenylene oxide sulfone), and the like); poly(sulfones); poly(vinyl acetates); copolymers of vinyl acetate (for example, poly(ethylene-co-vinyl acetate), copolymers in which at least some of the acetate groups have been hydrolyzed to provide various poly(vinyl alcohols) including poly(ethylene-co-vinyl alcohol), and the like); poly(phosphazenes); poly(vinyl esters); poly(vinyl ethers); poly(vinyl alcohols); polyaramids (for example, para-aramids such as poly(paraphenylene terephthalamide) and fibers sold under the trade designation “KEVLAR” by DuPont Co., Wilmington, Del., pulps of which are commercially available in various grades based on the length of the fibers that make up the pulp such as, for example, “KEVLAR 1F306” and “KEVLAR 1F694”, both of which include aramid fibers that are at least 4 mm in length; and the like); poly(carbonates); and the like; and combinations thereof. Preferred polymeric fibers include polyamides, polyolefins, polysulfones, and combinations thereof (more preferably, polyamides, polyolefins, and combinations thereof; most preferably, nylons, poly(ethylene), and combinations thereof).

Suitable inorganic fibers include those that comprise at least one inorganic material selected from glasses, ceramics, and combinations thereof. Useful inorganic fibers include fiberglasses (for example, E-glass, S-glass, and the like), ceramic fibers (for example, fibers made of metal oxides (such as alumina), silicon carbide, boron nitride, boron carbide, and the like), and the like, and combinations thereof. Useful ceramic fibers can be at least partially crystalline (exhibiting a discernible X-ray powder diffraction pattern or containing both crystalline and amorphous (glass) phases). Preferred inorganic fibers include fiberglasses and combinations thereof.

The fibers used to form the porous fibrous nonwoven matrix can be of a length and diameter that can provide a matrix having sufficient structural integrity and sufficient porosity for a particular application (for example, for a particular type of sample matrix). For example, lengths of at least about 0.5 mm, 1 mm, 2 mm, 3 mm, 4 mm, 6 mm, 8 mm, 10 mm, 15 mm, 20 mm, 25 mm, or even 30 mm (and combinations thereof), and diameters of at least about 10 μm (micrometer), 20 μm, 40 μm, or even 60 μm (and combinations thereof) can be useful. Preferred fiber lengths and diameters will vary, depending upon factors including the nature of the fiber and the type of application. For example, fibrillated poly(ethylene) can be useful in lengths of about 1 mm to about 3 mm, and non-fibrillated nylon can be useful in lengths of about 6 mm to about 12.5 mm, for a variety of sample matrices.

To facilitate entrapment of the concentration agent particles and/or to ensure a high surface area matrix, the fibers used to form the porous fibrous nonwoven matrix preferably comprise at least one fibrillated fiber (for example, in the form of a main fiber surrounded by many smaller attached fibrils). The main fiber generally can have a length in the range of about 0.5 mm to about 4 mm and a diameter of about 1 to about 20 micrometers. The fibrils typically can have a submicrometer diameter.

The porous fibrous nonwoven matrix can comprise two, three, four, or even more different types of fibers. For example, a nylon fiber can be added for strength and integrity, while fibrillated polyethylene can be added for entrapment of the particulates. If fibrillated and non-fibrillated fibers are used, generally the weight ratio of fibrillated fibers to non-fibrillated fibers can be at least about 1:2, 1:1, 2:1, 3:1, 5:1, or even 8:1. Regardless of the type(s) of fibers chosen, the amount of fiber in the resulting concentration device (in dry form) is preferably at least about 10, 12, 12.5, 14, 15, 18, 20, or even 22 percent by weight up to about 20, 25, 27, 30, 35, or even 40 percent by weight (based upon the total weight of all components of the concentration device).

Preferably, the porous fibrous nonwoven matrix further comprises at least one polymeric binder. Suitable polymeric binders include natural and synthetic polymeric materials that are relatively inert (exhibiting little or no chemical interaction with either the fibers or the concentration agent particles). Useful polymeric binders include polymeric resins (for example, in the form of powders and latexes), polymeric binder fibers, and the like, and combinations thereof. For at least some applications, preferred polymeric binders include polymeric binder fibers and combinations thereof. For other applications, polymeric resins and combinations thereof can be preferred polymeric binders.

Suitable polymeric resins include, but are not limited to, natural rubbers, neoprene, styrene-butadiene copolymers, acrylate resins, polyvinyl chloride, polyvinyl acetate, and the like, and combinations thereof. Preferred polymeric resins include acrylate resins and combinations thereof. Suitable polymeric binder fibers include adhesive-only type fibers (for example, Kodel™ 43UD fibers, available from Eastman Chemical Products, Kingsport, Tenn.), bicomponent fibers (for example, side-by-side forms such as Chisso ES polyolefin thermally bonded bicomponent fibers, available from Chisso Corporation, Osaka, Japan; sheath-core forms such as Melty™ Fiber Type 4080 bicomponent fibers having a polyester core and a polyethylene sheath, available from Unitika Ltd., Osaka, Japan; and the like), and the like, and combinations thereof. Preferred polymeric binder fibers include bicomponent fibers and combinations thereof (more preferably, sheath-core bicomponent fibers and combinations thereof).

Regardless of the type of polymeric binder used, the amount of binder in the resulting concentration device (in dry form) generally can be from about 3 weight percent to about 7 weight percent (preferably, about 5 weight percent), based upon the total weight of all components of the concentration device. Such amounts of polymeric binder generally can provide the porous fibrous nonwoven matrix with sufficient integrity for use in many applications, while not significantly coating the particles. Surprisingly, the amount of polymeric binder in the concentration device can be less than about 5, 4, 3, 2, or even 1 percent by weight, relative to the weight of the fibers in the concentration device.

In preferred embodiments of the concentration device, the polymeric binder does not substantially adhere to the particles. In other words, when the concentration device is examined by scanning electron microscopy, less than about 5, 4, 3, 2, or even 1 percent of the total surface area of the particles is covered with polymeric binder.

The concentration device used in the process of the invention can be prepared by a process comprising (a) providing a plurality of the above-described fibers; (b) providing a plurality of the above-described concentration agent particles; and (c) forming at least a portion of the plurality of fibers into a porous fibrous nonwoven matrix having at least a portion of the plurality of particles enmeshed therein. As mentioned above, the forming can be carried out by essentially any process that is capable of providing a fibrous nonwoven matrix (that is, a web or medium, other than a woven or knitted fabric, comprising interlaid fibers) having the concentration agent particles enmeshed therein. Useful processes include meltblowing, spunbonding, and other air laying techniques; carding; wet laying; and the like; and combinations thereof (preferably, air laying, wet laying, and combinations thereof; more preferably, wet laying).

Preferably, the forming is carried out by using a wet laying or “wetlaid” process comprising (a) forming a dispersion comprising the plurality of fibers, the plurality of particles (which can be added and dispersed along with the other components prior to carrying out other process steps or, if desired, can be added and dispersed later in the process but generally prior to removal of dispersing liquid), and at least one polymeric binder in at least one dispersing liquid (preferably, water); (b) at least partially depositing the polymeric binder onto at least a portion of the fibers; and (c) removing the dispersing liquid from the dispersion. In such a process, the fibers can be dispersed in the dispersing liquid to form a slurry. If desired, the fibers can comprise additives or chemical groups or moieties to assist in their dispersion. For example, polyolefin-based fibers can comprise maleic anhydride or succinic anhydride functionality, or, during the melt-processing of polyethylene fibers, a suitable surfactant can be added.

Deposition of the polymeric binder onto the fibers can be carried out either before or after the dispersing liquid removal or dewatering step, depending upon the nature of the polymeric binder. For example, when a polymeric latex is used as the polymeric binder, the polymeric latex can be precipitated onto the fibers before or after particle addition and prior todewatering. After the dewatering, heat can be applied to finish the dewatering and to set the resulting deposited latex. When polymeric binder fibers are used as the polymeric binder, dewatering can generally be carried out first, followed by heating to finish the dewatering and to melt the polymeric binder fibers (and thereby deposit polymeric binder on the fibers).

One or more adjuvants or additives can be used in preparing the concentration device. Useful adjuvants include process aids (for example, precipitation agents such as sodium aluminate and aluminum sulfate, which can aid in precipitating the polymeric binder onto the fibers), materials that can enhance the overall performance of the resulting concentration device, and the like. When used, the amounts of such adjuvants can range from more than zero up to about 2 weight percent (preferably, up to about 0.5 weight percent; based upon the total weight of the components of the concentration device), although their amounts are preferably kept as low as possible so as to maximize the amount of concentration agent particles that can be included.

In a preferred wetlaid process, the fibers (for example, chopped fibers) can be blended in a container in the presence of the dispersing liquid (for example, water, a water-miscible organic solvent such as an alcohol, or a combination thereof). The amount of shear used to blend the resulting mixture has not been found to affect the ultimate properties of the resulting concentration device, although the amount of shear introduced during blending is preferably relatively high. Thereafter, the particles, the polymeric binder, and an excess of a precipitation agent (for example, a pH adjusting agent such as alum) can be added to the container.

When the preferred wetlaid process is carried out by using hand-sheet methods known in the art, the order of addition of the three ingredients to the fiber dispersion has not been found to significantly affect the ultimate performance of the concentration device. Addition of the polymeric binder after addition of the particles, however, can provide a concentration device exhibiting somewhat greater adhesion of the particles to the fibers. When the preferred wetlaid process is carried out by using a continuous method, the three ingredients preferably are added in the listed order. (The following description is based on a hand-sheet method, although those skilled in the art can readily recognize how to adapt such a method to provide for a continuous process.)

After the particles, the polymeric binder, and the precipitation agent are added to the fiber-liquid slurry, the resulting mixture can be poured into a mold, the bottom of which can be covered by a screen. The dispersing liquid (preferably, water) can be allowed to drain from the mixture (in the form of a wet sheet) through the screen. After sufficient liquid has drained from the sheet, the wet sheet generally can be removed from the mold and dried by pressing, heating, or a combination of the two. Generally pressures of about 300 to about 600 kPa and temperatures of about 100 to about 200° C. (preferably, about 100 to about 150° C.) can be used in these drying processes. When polymeric binder fibers are used as the polymeric binder in the preferred wetlaid process, no precipitation agent is needed, and the applied heat can be used to melt the polymeric binder fibers.

The resulting dry sheet can have an average thickness of at least about 0.2, 0.5, 0.8, 1, 2, 4, or even 5 min up to about 5, 8, 10, 15, or even 20 mm. Up to about 100 percent of the dispersing liquid can be removed (preferably, up to about 90 percent by weight). Calendering can be used to provide additional pressing or fusing, if desired.

As mentioned above, the concentration agent particles can be microparticles. The microparticles can be entrapped in the porous fibrous nonwoven matrix through either chemical interactions (for example, chemical bonding) or physical interactions (for example, adsorption or mechanical entrapment), depending upon the nature of the fibers that are utilized. Preferred embodiments of the concentration device include those comprising at least one fibrillated fiber that can effect mechanical entrapment of the concentration agent particles. In one embodiment of the concentration device, the effective average diameter of the particles is at least about 175 times smaller than the uncalendered thickness of the resulting wetlaid sheet (preferably, at least about 250 times smaller than the uncalendered thickness of the sheet; more preferably, at least about 300 times smaller than the uncalendered thickness of the sheet).

Since the capacity and efficiency of the concentration device can vary according to the amount of concentration agent particles contained therein, relatively high particle loadings generally can be desirable. The amount of particles in the concentration device preferably can be at least about 20, 30, 40, 50, 60, 70, or even 80 weight percent (based upon the total weight of all components of the concentration device). The particles are entrapped in the porous fibrous nonwoven matrix and preferably distributed within it (more preferably, the particles are distributed essentially uniformly throughout the matrix).

The resulting concentration device can have controlled porosity (preferably, having a Gurley time of at least about 0.1 second (more preferably, at least about 2 to about 4 seconds; most preferably, at least about 4 seconds) for 100 mL of air). The basis weight of the concentration device (in the form of sheet material) can be in the range of about 250 to about 5000 g/m² (preferably, in the range of about 400 to about 1500 g/m²; more preferably, about 500 to about 1200 g/m²).

Generally the average pore size of the sheet material can be in the range of about 0.1 to about 10 micrometers, as measured by scanning electron microscopy (SEM). Void volumes in the range of about 20 to about 80 volume percent can be useful (preferably, about 40 to about 60 volume percent). The porosity of the sheet materials can be modified (increased) by including fibers of larger diameter or stiffness in the fiber mixture.

The sheet material can be flexible (for example, able to be rolled around a 0.75 inch (about 2 cm) diameter core). This flexibility can enable the sheet material to be pleated or rolled. The sheet material can have a relatively low back pressure (meaning that a relatively high volume of liquid can be relatively quickly passed through it without generating relatively high back pressure). (As used herein, “relatively low back pressure” refers to a differential back pressure of less than about 3 pounds per square inch (20.7 kPa), 2.5 (17.2), 2 (13.8), 1.5 (10.3), or even 1 pound per square inch (6.9 kPa) at a 3 mL/cm² flowrate, wherein the flowrate is based on the frontal surface area of the sheet material.)

The uncalendered sheet material can be cut to a desired size and used to carry out the concentration process of the invention. If desired (for example, when a significant pressure drop across the sheet is not a concern), the sheet material can be calendered to increase its tensile strength prior to use. When the sheet material is to be pleated, drying and calendering preferably can be avoided.

A single layer of sheet material can be effective in carrying out the concentration process of the invention. Multiple layers can be used, if desired, to provide greater concentration capacity.

A significant advantage of the porous fibrous nonwoven matrix of the concentration device is that very small concentration agent particle sizes (10 μm or smaller) and/or concentration agent particles with a relatively broad size distribution can be employed. This allows for excellent one-pass kinetics, due to increased surface area/mass ratios and, for porous particles, minimized internal diffusion distances. Because of the relatively low pressure drops, a minimal driving force (such as gravity or a vacuum) can be used to pull a sample through the concentration device, even when small concentration agent particle sizes are employed.

If desired, the concentration device can further comprise one or more other components such as, for example, one or more pre-filters (for example, to remove relatively large food particles from a sample prior to passage of the sample through the porous matrix), a support or base for the porous matrix (for example, in the form of a frit or grid), a manifold for applying a pressure differential across the device (for example, to aid in passing a sample through the porous matrix), and/or an external housing (for example, a disposable cartridge to contain and/or protect the porous matrix).

### Sample

The process of the invention can be applied to a variety of different types of samples, including, but not limited to, medical, environmental, food, feed, clinical, and laboratory samples, and combinations thereof. Medical or veterinary samples can include, for example, cells, tissues, or fluids from a biological source (for example, a human or an animal) that are to be assayed for clinical diagnosis. Environmental samples can be, for example, from a medical or veterinary facility, an industrial facility, soil, a water source, a food preparation area (food contact and non-contact areas), a laboratory, or an area that has been potentially subjected to bioterrorism. Food processing, handling, and preparation area samples are preferred, as these are often of particular concern in regard to food supply contamination by bacterial pathogens.

Samples obtained in the form of a liquid or in the form of a dispersion or suspension of solid in liquid can be used directly, or can be concentrated (for example, by centrifugation) or diluted (for example, by the addition of a buffer (pH-controlled) solution). Samples in the form of a solid or a semi-solid can be used directly or can be extracted, if desired, by a method such as, for example, washing or rinsing with, or suspending or dispersing in, a fluid medium (for example, a buffer solution). Samples can be taken from surfaces (for example, by swabbing or rinsing). Preferably, the sample is a fluid (for example, a liquid, a gas, or a dispersion or suspension of solid or liquid in liquid or gas).

Examples of samples that can be used in carrying out the process of the invention include foods (for example, fresh produce or ready-to-eat lunch or “deli” meats), beverages (for example, juices or carbonated beverages), water (including potable water), and biological fluids (for example, whole blood or a component thereof such as plasma, a platelet-enriched blood fraction, a platelet concentrate, or packed red blood cells; cell preparations (for example, dispersed tissue, bone marrow aspirates, or vertebral body bone marrow); cell suspensions; urine, saliva, and other body fluids; bone marrow; lung fluid; cerebral fluid; wound exudate; wound biopsy samples; ocular fluid; spinal fluid; and the like), as well as lysed preparations, such as cell lysates, which can be formed using known procedures such as the use of lysing buffers, and the like. Preferred samples include foods, beverages, water, biological fluids, and combinations thereof (with foods, beverages, water, and combinations thereof being more preferred, and with water being most preferred).

Sample volume can vary, depending upon the particular application. For example, when the process of the invention is used for a diagnostic or research application, the volume of the sample can typically be in the microliter range (for example, 10 microliters or greater). When the process is used for a food pathogen testing assay or for potable water safety testing, the volume of the sample can typically be in the milliliter to liter range (for example, 100 milliliters to 3 liters). In an industrial application, such as bioprocessing or pharmaceutical formulation, the volume can be tens of thousands of liters.

The process of the invention can isolate microorganisms from a sample in a concentrated state and can also allow the isolation of microorganisms from sample matrix components that can inhibit detection procedures that are to be used. In all of these cases, the process of the invention can be used in addition to, or in replacement of, other methods of cellular analyte or microorganism concentration. Thus, optionally, cultures can be grown from samples either before or after carrying out the process of the invention, if additional concentration is desired. Such cultural enrichment can be general or primary (so as to enrich the concentrations of most or essentially all microorganisms) or can be specific or selective (so as to enrich the concentration(s) of one or more selected microorganisms only).

### Contacting

The process of the invention can be carried out by any of various known or hereafter-developed methods of providing contact between two materials. For example, the concentration device can be added to the sample, or the sample can be added to the concentration device. The concentration device can be immersed in a sample, a sample can be poured onto the concentration device, a sample can be poured into a tube or well containing the concentration device, or, preferably, a sample can be passed over or through (preferably, through) the concentration device (or vice versa). Preferably, the contacting is carried out in a manner such that the sample passes through at least one pore of the porous fibrous nonwoven matrix (preferably, through at least one through pore).

The concentration device and the sample can be combined (using any order of addition) in any of a variety of containers or holders (optionally, a capped, closed, or sealed container; preferably, a column, a syringe barrel, or another holder designed to contain the device with essentially no sample leakage). Suitable containers for use in carrying out the process of the invention will be determined by the particular sample and can vary widely in size and nature. For example, the container can be small, such as a 10 microliter container (for example, a test tube or syringe) or larger, such as a 100 milliliter to 3 liter container (for example, an Erlenmeyer flask or an annular cylindrical container).

The container, the concentration device, and any other apparatus or additives that contact the sample directly can be sterilized (for example, by controlled heat, ethylene oxide gas, or radiation) prior to use, in order to reduce or prevent any contamination of the sample that might cause detection errors. The amount of concentration agent in the concentration device that is sufficient to capture or concentrate the microorganisms of a particular sample for successful detection will vary (depending upon, for example, the nature and form of the concentration agent and device and the volume of the sample) and can be readily determined by one skilled in the art.

Contacting can be carried out for a desired period (for example, for sample volumes of several liters or for processes involving multiple passes through the concentration device, up to about 60 minutes of contacting can be useful; preferably, about 15 seconds to about 10 minutes or longer; more preferably, about 15 seconds to about 5 minutes; most preferably, about 15 seconds to about 2 minutes). Contact can be enhanced by mixing (for example, by stirring, by shaking, or by application of a pressure differential across the device to facilitate passage of a sample through its porous matrix) and/or by incubation (for example, at ambient temperature), which are optional but can be preferred, in order to increase microorganism contact with the concentration device.

Preferably, contacting can be effected by passing a sample at least once (preferably, only once) through the concentration device (for example, by pumping). Essentially any type of pump (for example, a peristaltic pump) or other equipment for establishing a pressure differential across the device (for example, a syringe or plunger) can be utilized. Useful flow rates will vary, depending upon such factors as the nature of the sample matrix and the particular application.

For example, sample flow rates through the device of up to about 100 milliliters per minute or more can be effective. Preferably, for samples such as beverages and water, flow rates of about 10-20 milliliters per minute can be utilized. For pre-filtered or otherwise clarified food samples, flow rates of about 6 milliliters per minute (1.5 milliliters per 15 seconds) can be useful. Longer contact times and slower flow rates can be useful for more complex sample matrices such as ground beef or turkey.

A preferred contacting method includes such passing of a sample through the concentration device (for example, by pumping). If desired, one or more additives (for example, lysis reagents, bioluminescence assay reagents, nucleic acid capture reagents (for example, magnetic beads), microbial growth media, buffers (for example, to moisten a solid sample), microbial staining reagents, washing buffers (for example, to wash away unbound material), elution agents (for example, serum albumin), surfactants (for example, Triton™ X-100 nonionic surfactant available from Union Carbide Chemicals and Plastics, Houston, Tex.), mechanical abrasion/elution agents (for example, glass beads), adsorption buffers (for example, the same buffer used for preparing the above-mentioned adsorption buffer-modified concentration agent or a different buffer), and the like) can be included in the combination of concentration device and sample during contacting.

The process of the invention can optionally further comprise separating the resulting target cellular analyte-bound concentration device and the sample. Separation can be carried out by numerous methods that are well-known in the art (for example, by pumping, decanting, or siphoning a fluid sample, so as to leave the target cellular analyte-bound concentration device in the container or holder utilized in carrying out the process). It can also be possible to isolate or separate captured target cellular analytes (target microorganisms or one or more components thereof) from the concentration device after sample contacting (for example, by passing an elution agent or a lysis agent over or through the concentration device).

The process of the invention can be carried out manually (for example, in a batch-wise manner) or can be automated (for example, to enable continuous or semi-continuous processing).

### Detection

A variety of microorganisms can be concentrated and detected by using the process of the invention, including, for example, bacteria, fungi, yeasts, protozoans, viruses (including both non-enveloped and enveloped viruses), bacterial endospores (for example, Bacillus (including Bacillus anthracis, Bacillus cereus, and Bacillus subtilis) and Clostridium (including Clostridium botulinum, Clostridium difficile, and Clostridium perfringens)), and the like, and combinations thereof (preferably, bacteria, yeasts, viruses, bacterial endospores, fungi, and combinations thereof; more preferably, bacteria, yeasts, bacterial endospores, fungi, and combinations thereof; even more preferably, bacteria, yeasts, fungi, and combinations thereof; still more preferably, gram-negative bacteria, gram-positive bacteria, yeasts, fungi, and combinations thereof; most preferably, gram-negative bacteria, gram-positive bacteria, yeasts, and combinations thereof). The process has utility in the detection of pathogens, which can be important for food safety or for medical, environmental, or anti-terrorism reasons. The process can be particularly useful in the detection of pathogenic bacteria (for example, both gram negative and gram positive bacteria), as well as various yeasts and molds (and combinations of any of these).

Genera of target microorganisms to be detected include, but are not limited to, Listeria, Escherichia, Salmonella, Campylobacter, Clostridium, Helicobacter, Mycobacterium, Staphylococcus, Shigella, Enterococcus, Bacillus, Neisseria, Shigella, Streptococcus, Vibrio, Yersinia, Bordetella, Borrelia, Pseudomonas, Saccharomyces, Candida, and the like, and combinations thereof. Samples can contain a plurality of microorganism strains, and any one strain can be detected independently of any other strain. Specific microorganism strains that can be targets for detection include Escherichia coli, Yersinia enterocolitica, Yersinia pseudotuberculosis, Vibrio cholerae, Vibrio parahaemolyticus, Vibrio vulnificus, Listeria monocytogenes (for which Listeria innocua is a surrogate), Staphylococcus aureus, Salmonella enterica, Saccharomyces cerevisiae, Candida albicans, Staphylococcal enterotoxin ssp, Bacillus cereus, Bacillus anthracis, Bacillus atrophaeus, Bacillus subtilis, Clostridium perfringens, Clostridium botulinum, Clostridium difficile, Enterobacter sakazakii, human-infecting non-enveloped enteric viruses for which Escherichia coli bacteriophage is a surrogate, Pseudomonas aeruginosa, and the like, and combinations thereof (preferably, Staphylococcus aureus, Listeria monocytogenes (for which Listeria innocua is a surrogate), Salmonella enterica, Saccharomyces cerevisiae, Bacillus subtilis, Pseudomonas aeruginosa, Escherichia coli, human-infecting non-enveloped enteric viruses for which Escherichia coli bacteriophage is a surrogate, and combinations thereof; more preferably, Staphylococcus aureus, Listeria monocytogenes (for which Listeria innocua is a surrogate), Pseudomonas aeruginosa, and combinations thereof).

Microorganisms that have been captured or bound (for example, by adsorption or by sieving) by the concentration device can be detected by essentially any desired method that is currently known or hereafter developed. Such methods include, for example, culture-based methods (which can be preferred when time permits), microscopy (for example, using a transmitted light microscope or an epifluorescence microscope, which can be used for visualizing microorganisms tagged with fluorescent dyes) and other imaging methods, immunological detection methods, and genetic detection methods. The detection process following microorganism capture optionally can include washing to remove sample matrix components, slicing or otherwise breaking up the porous fibrous nonwoven matrix of the concentration device, staining, boiling or using elution buffers or lysis agents to release cellular analyte from the concentration device, or the like.

Immunological detection is detection of an antigenic material derived from a target organism, which is commonly a biological molecule (for example, a protein or proteoglycan) acting as a marker on the surface of bacteria or viral particles. Detection of the antigenic material typically can be by an antibody, a polypeptide selected from a process such as phage display, or an aptamer from a screening process.

Immunological detection methods are well-known and include, for example, immunoprecipitation and enzyme-linked immunosorbent assay (ELISA). Antibody binding can be detected in a variety of ways (for example, by labeling either a primary or a secondary antibody with a fluorescent dye, with a quantum dot, or with an enzyme that can produce chemiluminescence or a colored substrate, and using either a plate reader or a lateral flow device).

Detection can also be carried out by genetic assay (for example, by nucleic acid hybridization or primer directed amplification), which is often a preferred method. The captured or bound microorganisms can be lysed to render their genetic material available for assay. Lysis methods are well-known and include, for example, treatments such as sonication, osmotic shock, high temperature treatment (for example, from about 50° C. to about 100° C.), and incubation with an enzyme such as lysozyme, glucolase, zymolose, lyticase, proteinase K, proteinase E, and viral enolysins.

Many commonly-used genetic detection assays detect the nucleic acids of a specific microorganism, including the DNA and/or RNA. The stringency of conditions used in a genetic detection method correlates with the level of variation in nucleic acid sequence that is detected. Highly stringent conditions of salt concentration and temperature can limit the detection to the exact nucleic acid sequence of the target. Thus microorganism strains with small variations in a target nucleic acid sequence can be distinguished using a highly stringent genetic assay. Genetic detection can be based on nucleic acid hybridization where a single-stranded nucleic acid probe is hybridized to the denatured nucleic acids of the microorganism such that a double-stranded nucleic acid is produced, including the probe strand. One skilled in the art will be familiar with probe labels, such as radioactive, fluorescent, and chemiluminescent labels, for detecting the hybrid following gel electrophoresis, capillary electrophoresis, or other separation method.

Particularly useful genetic detection methods are based on primer directed nucleic acid amplification. Primer directed nucleic acid amplification methods include, for example, thermal cycling methods (for example, polymerase chain reaction (PCR), reverse transcriptase polymerase chain reaction (RT-PCR), and ligase chain reaction (LCR)), as well as isothermal methods and strand displacement amplification (SDA) (and combinations thereof; preferably, PCR or RT-PCR). Methods for detection of the amplified product are not limited and include, for example, gel electrophoresis separation and ethidium bromide staining, as well as detection of an incorporated fluorescent label or radio label in the product. Methods that do not require a separation step prior to detection of the amplified product can also be used (for example, real-time PCR or homogeneous detection).

Bioluminescence detection methods are well-known and include, for example, adensosine triphosphate (ATP) detection methods including those described in U.S. Pat. No. 7,422,868 (Fan et al.), the descriptions of which are incorporated herein by reference. Other luminescence-based detection methods can also be utilized.

Since the process of the invention is non-strain specific, it provides a general capture system that allows for multiple microorganism strains to be targeted for assay in the same sample. For example, in assaying for contamination of food samples, it can be desired to test for Listeria monocytogenes, Escherichia coli, and Salmonella all in the same sample. A single capture step can then be followed by, for example, PCR or RT-PCR assays using specific primers to amplify different nucleic acid sequences from each of these microorganism strains. Thus, the need for separate sample handling and preparation procedures for each strain can be avoided.

### Diagnostic Kit

A diagnostic kit for use in carrying out the concentration process of the invention comprises (a) at least one above-described concentration device; and (b) at least one testing container or testing reagent (preferably, a sterile testing container or testing reagent) for use in carrying out the concentration process of the invention. Preferably, the diagnostic kit further comprises instructions for carrying out the process.

Useful testing containers or holders include those described above and can be used, for example, for contacting, for incubation, for collection of eluate, or for other desired process steps. Useful testing reagents include microorganism culture or growth media, lysis agents, elution agents, buffers, luminescence detection assay components (for example, luminometer, lysis reagents, luciferase enzyme, enzyme substrate, reaction buffers, and the like), genetic detection assay components, and the like, and combinations thereof. A preferred lysis agent is a lytic enzyme or chemical supplied in a buffer, and preferred genetic detection assay components include one or more primers specific for a target microorganism. The kit can optionally further comprise sterile forceps or the like.

### Filter Media

In other embodiments, the present disclosure provides a filter media for removing microbial contaminants or pathogens from a sample (e.g., water). Filter media suitable for use in accordance with the present disclosure include those that comprise (a) a porous fibrous nonwoven matrix and (b) a plurality of the above-described concentration agent particles, the particles being enmeshed in the porous fibrous nonwoven matrix. Such filter media can be prepared by essentially the same processes, and include essentially the same materials, as those described above with respect to concentration agents and concentration devices.

### EXAMPLES

Objects and advantages of this invention are further illustrated by the following examples, but the particular materials and amounts thereof recited in these examples, as well as other conditions and details, should not be construed to unduly limit this invention. All parts, percentages, ratios, and so forth, in the following examples are by weight, unless noted otherwise. Solvents and other reagents were obtained from Sigma-Aldrich Chemical Company, Milwaukee, Wis., unless specified differently. All microorganism cultures were purchased from The American Type Culture Collection (ATCC; Manassas, Va.). Experimental results are an average of 2 tests, unless otherwise stated. Overnight cultures were prepared by streaking selected microorganisms on Tryptic Soy Agar plates and then incubating the plates at 37° C. overnight. All microorganism counts were performed according to standard microbiological counting procedures for colony forming units, and counts are approximate numbers.

### Materials

Particulate Concentration Agent 1 (hereinafter, “Particle 1”)—diatomaceous earth surface-modified by deposition of iron oxide (prepared essentially as described below)
Particulate Concentration Agent 2 (hereinafter, “Particle 2”)—diatomaceous earth surface-modified by deposition of titanium dioxide (prepared essentially as described below)
BHI Broth—Difco™ Bovine Heart Infusion Broth general-purpose growth medium from Becton Dickinson, Sparks, Md., prepared at 3.7 weight percent (wt %) concentration according to the manufacturer's instructions
Buffer solution—Butterfield's Buffer, pH 7.2±0.2; monobasic potassium phosphate buffer solution; VWR Catalog Number 83008-093; VWR, West Chester, Pa.
Tryptic Soy Agar plate—Difco™ Tryptic Soy Agar obtained from Becton Dickinson, Sparks, Md., prepared at 3 weight percent (wt %) according to the manufacturer's instructions using Difco™ Tryptic Soy Broth, Becton Dickinson, Sparks, Md.
MOX plate—Oxford Medium, Modified for Listeria, agar-based growth medium obtained from Hardy Diagnostics, Santa Maria, Calif.
AC plate—3M™ Petrifilm™ Aerobic Count Plate (a flat film culture device comprising dry, rehydratable culture medium); 3M Company, St. Paul, Minn.
PIA plate—Pseudomonas Isolation Agar made by Teknova; purchased from VWR, West Chester, Pa.
C-agar plate—BBL™ CHROMagar™ Staph aureus plate (agar-based growth medium) made by Becton Dickinson; purchased from VWR, West Chester, Pa.
Elisa Assay—3M™ TECRA™ Listeria Visual Immunoassay kit; 3M Company, St. Paul, Minn.
Vacuum filtration apparatus—A 1000 mL flask, having a side vacuum port, was fitted with a sintered stopper that served as the support for a porous fibrous nonwoven matrix or filter. The support area was sized to hold a 48 mm diameter circular disk of the porous fibrous nonwoven matrix. An open-ended collection cylinder (100 mL capacity), having flanged rims around the top and bottom of the cylinder, was clamped to the flask with the stopper secured between them. A flexible hose connected the flask to a faucet equipped with a vacuum port to provide a vacuum. The apparatus was sterilized by autoclaving at 121° C. for 15 minutes before each period of use and, during use, was rinsed with 70 weight percent (wt %) ethanol and distilled water after filtration of each sample.
Filter holder—13 mm diameter Swinnex™ filter holder; Millipore Corp., Bedford, Mass.
Stomacher and stomacher bags—Stomacher™ 400 Circulator laboratory blender and Stomacher™ polyethylene filter bags, Seward Corp., Norfolk, UK; purchased from VWR, West Chester, Pa.

### Terms

Porous fibrous nonwoven matrix—may also be referred to as a dry felt, a pad, a matrix, or a filter in the following examples and comparative examples
Concentration of liquid—A microorganism-containing 100 mL or 250 mL liquid sample was passed through a porous fibrous nonwoven matrix or filter, and the microorganisms were collected or concentrated by the filter. The filter was then analyzed (for example, plated or otherwise assayed) using 1 or 2 mL of added buffer or water. Thus, the microorganisms were concentrated from 100 mL or 250 mL of liquid to 1 or 2 mL of liquid.
CFUs—colony-forming units
Filtrate count—the count of microorganism colonies in a filtrate
Pre-filtration count—the count of microorganism colonies in a pre-filtration sample (that is, the microorganism count of an unconcentrated sample)
MCE—Microorganism Capture Efficiency (or Binding Efficiency) of a porous fibrous nonwoven matrix is an assessment of how well the matrix captures microorganisms. The MCE, in percent (%), was determined by the following formula:

MCE=100−[(Filtrate count/Pre-filtration count)×100]
0.5 McFarland Standard—a turbidity standard comprising dispersed microorganisms, prepared using a DensiCHEK™ densitometer from bioMerieux, Inc., Durham, N.C.

### Preparation of Surface-Modified Diatomaceous Earth Particulate Concentration Agents

Kieselguhr (diatomaceous earth) was purchased from Alfa Aesar (A Johnson Matthey Company, Ward Hill, Mass.) as a white powder (325 mesh; all particles less than 44 micrometers in size). This material was shown by X-ray diffraction (XRD) to contain amorphous silica along with crystalline α-cristobalite and quartz.

Particulate concentration agents comprising two different surface modifiers (namely, titanium dioxide and ferric oxide) were prepared by surface treating the diatomaceous earth in the manner described below:

### Deposition of Titanium Dioxide

A 20 weight percent titanium (IV) oxysulfate dehydrate solution was prepared by dissolving 20.0 g of TiO(SO₄).2H₂O (Noah Technologies Corporation, San Antonio, Tex.) in 80.0 g of deionized water with stirring. 50.0 g of this solution was mixed with 175 mL of deionized water to form a titanium dioxide precursor compound solution. A dispersion of diatomaceous earth was prepared by dispersing 50.0 g of diatomaceous earth in 500 mL of deionized water in a large beaker with rapid stirring. After heating the diatomaceous earth dispersion to about 80° C., the titanium dioxide precursor compound solution was added dropwise while rapidly stirring over a period of about 1 hour. After the addition, the beaker was covered with a watch glass and its contents heated to boiling for 20 minutes. An ammonium hydroxide solution was added to the beaker until the pH of the contents was about 9. The resulting product was washed by settling/decantation until the pH of the wash water was neutral. The product was separated by filtration and dried overnight at 100° C.

A portion of the dried product was placed into a porcelain crucible and calcined by heating from room temperature to 350° C. at a heating rate of about 3° C. per minute and then held at 350° C. for 1 hour.

### Deposition of Iron Oxide

Iron oxide was deposited onto diatomaceous earth using essentially the above-described titanium dioxide deposition process, with the exception that a solution of 20.0 g of Fe(NO₃)₃.9H₂O (J. T. Baker, Inc., Phillipsburg, N.J.) dissolved in 175 mL of deionized water was substituted for the titanyl sulfate solution. A portion of the resulting iron oxide-modified diatomaceous earth was similarly calcined to 350° C. for further testing.

### Examples 1-2 and Comparative Example C1

### Preparation of Concentration Devices 1, 2, and C1

A fiber premix was prepared by first blending 30 g of 1 denier fibrillated polyethylene fibers (FYBREL™ 620 fibers; Minifibers, Inc., Johnson City, Tenn.) and 4 L of cold tap water in a 4 L blender (Waring Commercial Heavy Duty Blender, Model 37BL84) at medium speed for 30 seconds. The fibers were uniformly dispersed in the water with no clumps or nits at this point, and 6 g of 6 denier 0.25 inch long chopped nylon fibers (Minifibers, Inc., Johnson City, Tenn.) and 6 g of long glass fibers (Micro-Strand 106-475 Glass fiberglass; Schuller, Inc., Denver, Colo.) were then added to the fiber dispersion and blended at low speed for 30 seconds.

A matrix composition was prepared by adding 1000 mL of the resulting fiber premix to a 4 L stainless steel beaker and mixing with an impeller mixer (Fisher Scientific Stedfast Stirrer model SL2400; available from VWR, West Chester, Pa.) at a speed setting of 4 for 5 minutes. Then a dispersion of 1.0 g of latex binder (50 weight percent solids vinyl acetate emulsion; Airflex 600BP, Air Products Polymers, Allentown, Pa.) in about 25 mL of tap water in a 50 mL beaker was added to the mixed fiber premix, followed by the addition of another 25 mL water from rinsing the beaker. After mixing the resulting combination for 2 minutes, 2.0 g of flocculant (MP 9307 Flocculant (believed to be an aqueous solution of a copolymer of dimethylamine and epichlorohydrin), Midsouth Chemical Co., Inc., Riggold, La.) was pre-dispersed in about 25 mL of water and then added to the combination, followed by the addition of another 25 mL of rinse water from the beaker. The latex binder crashed out of solution onto the fibers, and the liquid phase of the matrix composition changed from cloudy to substantially clear. For Example 1, 10.0 g of Particle 1 was added to the resulting composition and vortexed for 1 minute. For Example 2, 10.0 g of Particle 2 was added to the resulting composition and vortexed for 1 minute. Comparative Example C1 was prepared in the same manner with no particles added.

A felt was prepared using a TAPPI™ pad maker apparatus (Williams Apparatus, Watertown, N.Y.). The apparatus had an enclosed box measuring about 20 centimeters (8 inches) square and 20 centimeters (8 inches) deep, with a fine mesh screen near the bottom and a drain valve below the screen. The box was filled with tap water to a height of about 1 cm above the screen. The matrix composition was poured into the box, and the valve was opened immediately, creating a vacuum that pulled the water out of the box. The resulting wetlaid felt was approximately 3 mm thick.

The wetlaid felt was transferred from the apparatus onto a sheet of blotter paper (20 centimeters by 20 centimeters (8 inches by 8 inches), 96-pound white paper, Anchor Paper, St. Paul, Minn.). The felt was sandwiched between 2-3 layers of blotter paper and pressed between 2 reinforced screens in an air-powered press set at 413 kPa (60 psi; calculated to be about 82.7 kPa (12 psi) pressure exerted on the felt) for 1-2 minutes until no further water was observed being expelled. The pressed felt was then transferred onto a fresh sheet of blotter paper and placed in an oven set at 125° C. for approximately 30 minutes to remove residual water and cure the latex binder to form a porous fibrous nonwoven matrix.

### Examples 3-4 and Comparative Example C2

### Testing of Concentration Devices 1, 2, and C1

A Listeria innocua (ATCC 33090) colony from an overnight streak culture was inoculated into 5 mL BHI Broth and incubated at 30° C. for 18-20 hours. The resulting culture, containing 10⁸ CFUs/mL, was diluted in buffer solution and inoculated into 100 mL of BHI Broth to provide a bacterial suspension containing 10⁵CFUs/mL (2×10⁷CFUs total). Circular disks (48 mm diameter) were cut from sheets of the porous fibrous nonwoven matrices of Examples 1, 2, and C1, and sterilized at 121° C. for 15 minutes. A disk from Example 1 was inserted into the vacuum filtration apparatus described above, and 100 mL of the bacterial suspension was poured through the disk in the apparatus until the entire sample passed through the disk. The process was repeated with a disk from Example 2. A disk from Comparative Example C1 was tested using a bacterial suspension containing 1×10⁷ CFUs total.

Two 100 microliter volumes of each of the resulting filtrates and of a pre-filtration control were diluted 1:10, 1:100, and 1:1000, plated onto MOX agar plates, and incubated at 37° C. for 18-20 hours. Colonies were counted manually, and Microorganism Capture Efficiency (MCE) was calculated. Results are shown in Table 1.

TABLE 1

| | Example No. | | |
|---|---|---|---|
| | C2 | 3 | 4 |
| Concentration Device | C1 | 1 | 2 |
| Microorganism Capture | less than 1 | 90 | 92 |
| Efficiency (%) | | | |

A 100-fold concentration (concentration of sample from 100 mL to 1 mL) was observed for all samples.

After filtering, the disks were removed from the apparatus using sterilized forceps and stored in sterile culture dishes until tested. The disks were cut with sterilized scissors and placed in sterile 50 mL polypropylene centrifuge tubes containing 2 mL of buffer solution for boiling. The samples were then processed using an Elisa Assay according to the manufacturer's instructions. The resulting absorbance (in absorbance units) was read from a spectrophotometer (SpectraMax™ M5 from Molecular Devices, Sunnyvale, Calif.) at a wavelength of 414 nanometers (A₄₁₄). Results are shown in Table 2.

TABLE 2

| | Example No. | | |
|---|---|---|---|
| | C2 | 3 | 4 |
| Concentration Device or | Negative Control of | 1 | 2 |
| Control | Elisa Assay | | |
| Absorbance (A₄₁₄) | 0.005 | 0.265 | 0.095 |
| Absorbance Magnitude | 1X | 53X | 19X |
| Relative to Control | | | |

The data in Table 2 show that greater absorbance (relative to the negative control of the Elisa Assay) was delivered by Examples 1 and 2, even though the disks of Examples 1 and 2 were boiled in 2 mL of buffer solution instead of 1 mL per the Elisa Assay instructions.

### Examples 5-6 and Comparative Example C3

### Testing of Concentration Devices 1, 2, and Commercial Nylon Filter

Ground turkey (labeled 12% fat) was purchased from a local grocery store. 11 g of the ground turkey was placed in a sterile stomacher bag and blended with 99 mL buffer solution in a stomacher at a speed of 200 revolutions per minute (rpm) for 1 minute. The blended sample was poured into the vacuum filtration apparatus containing a 48 mm disk of the matrix of Example 1 (for Example 5). The sample was filtered with vacuum from the water faucet until flow through the disk stopped, an indication that the disk was plugged. The procedure was repeated with a disk from Example 2 (for Example 6) and with a 0.45 micron nylon filter (for Comparative Example C3) obtained from 3M Purification, Inc., St. Paul, Minn. The total sample volume and the volume of the sample that passed through the disk prior to clogging are shown in Table 3 below.

TABLE 3

| Example | Concentration | Sample Volume Passed | Total Sample |
|---|---|---|---|
| No. | Device | Through Device (mL) | Volume (mL) |
| 5 | 1 | 7 | 100 |
| 6 | 2 | 8 | 100 |
| C3 | Commercial | 2.5 | 100 |
| | Nylon Filter | | |

The data in Table 3 show that the disks of Examples 5 and 6 had better resistance to plugging than the standard microbiology filter of Comparative Example C3, when processed using negative pressure.

### Examples 7-8 and Comparative Examples C4-C6

### Testing of Concentration Devices 1 and 2 and Comparison with Particulate Concentration Agents Alone (Particles 1 and 2)

An overnight culture of Listeria monocytogenes (ATCC 51414) was used to prepare a 0.5 McFarland Standard in 3 mL BHI Broth. The resulting bacterial stock containing 10⁸ CFUs/mL was serially diluted in BHI broth to obtain a bacterial suspension containing 10³ CFUs/mL.

Circular disks measuring 14 mm in diameter were die punched from the matrices of Examples 1 and 2 and Comparative Example C1. A disk from the matrix of Example 1 (for Example 7) was inserted into a 13 mm diameter filter holder. A 1.5 mL volume of the bacterial suspension, delivered to the filter holder through a 3 cubic centimeter (cc) syringe, passed through the disk in 20 seconds. The procedure was repeated with disks from the matrices of Example 2 (for Example 8) and Comparative Example C1 (for Comparative Example C6).

The resulting filtrates were plated in 100 microliter volumes on MOX plates in the following manner. The disks were each removed from the filter holder using surface-sterilized forceps and also plated on MOX plates with 100 microliters of buffer solution. The plates were incubated at 37° C. for 18-20 hours. The resulting microorganism colonies were counted manually.

For Comparative Example C4, a 20 mg quantity of Particle 1 was mixed with 1.1 mL of the bacterial suspension in a sterile 5 mL polypropylene tube (BD Falcon™, Becton Dickinson, Franklin Lakes, N.J.; obtained from VWR, West Chester, Pa.). A second sample was prepared in the same manner using 20 mg of Particle 2 (for Comparative Example C5). The tubes were capped and placed on a rocking platform (Thermolyne Vari Mix™ rocking platform; Barnstead International, Iowa) rocking at 14 cycles per minute for 20 seconds. Then the tubes were transferred to a test tube stand for one minute, after which most of the particles had settled to the bottom of the tubes. A volume of 100 microliters of the resulting supernatant, containing suspended particles, was plated on MOX plates and processed essentially as described above for the filtrates and disks. A volume of 100 microliters of the bacterial suspension was also plated and incubated in the same manner as a control (that is, pre-filtration) sample. The colony count for the control was 2500. Microorganism Capture Efficiency (MCE) was calculated based on colony counts from the filtrates and from the supernatants. Results are shown in Table 4 below.

TABLE 4

| | Concentration Device or | Microorganism Capture |
|---|---|---|
| Example No. | Agent | Efficiency (%) |
| C4 | Particle 1 | 59 |
| 7 | Concentration Device 1 | 98 |
| C5 | Particle 2 | 73 |
| 8 | Concentration Device 2 | 99 |
| C6 | Concentration Device C1 | 80 |

### Examples 9-10 and Comparative Example C7

### Testing of Concentration Devices 1 and 2 and a Commercial Polycarbonate Filter Membrane

Frozen ground beef (labeled 15% fat) was purchased from a local grocery store, and 11 g of thawed ground beef was blended with 99 mL of buffer solution in a sterile stomacher bag, and processed in a stomacher at 230 rpm for 30 seconds. A volume of 10 mL of blended beef was delivered in a 10 cc syringe to a 14 mm diameter disk of the matrix from Example 1 in a filter holder (for Example 9). A disk of the matrix from Example 2 and a commercial filter membrane (Whatman 14 mm diameter, 0.22 micron polycarbonate filter membrane purchased from VWR, West Chester, Pa.) were also tested as Example 10 and Comparative Example C7, respectively. The volume of blended beef passing through the disk or membrane prior to plugging and flow stoppage, and the time period of passage prior to plugging and flow stoppage, were recorded and are shown in Table 5.

TABLE 5

| | Example No. | | |
|---|---|---|---|
| | 9 | 10 | C7 |
| Concentration Device | 1 | 2 | Commercial Polycarbonate |
| | | | Filter Membrane |
| Sample Volume Passed | 4.5 | 8 | 0.5 |
| Through Device (mL) | | | |
| Passage Time (seconds) | 90 | 120 | 60 |

### Examples 11-14

### Testing of Concentration Devices 1 and 2

An overnight culture of Pseudomonas aeruginosa (ATCC 9027) was used to make a 0.5 McFarland Standard in 3 mL of filtered distilled deionized water (18 megaohm water obtained from a Milli-Q™ Gradient deionization system; Millipore Corporation, Bedford, Mass.). The resulting bacterial stock, containing 10⁸CFUs/mL, was serially diluted in the same water to obtain a P. aeruginosa suspension containing 10² CFUs/mL. A bacterial suspension of Staphylococcus aureus (ATCC 6538) was prepared using the same procedure.

A 1 mL volume of the P. aeruginosa suspension was filtered through a 13 mm disk from Example 1 (for Example 11) in a filter holder, essentially as described above for Examples 7-8. The resulting filtrate was plated on an AC plate according to the manufacturer's instructions. The disk was removed from the filter holder with sterilized forceps and plated on a PIA plate with 100 microliters of buffer solution. The filtration procedure was repeated using a disk from Example 2 (for Example 13).

The filtration procedure was then repeated using the S. aureus suspension and disks from Examples 1 and 2 (for Examples 12 and 14, respectively). The resulting filtrates were plated on AC plates, and the used disks were plated on C-agar plates with 100 microliters of buffer solution.

All of the plates were incubated at 37° C. for 18-20 hours, and the resulting colonies were counted manually and Microorganism Capture Efficiencies calculated. All of the plates showed growth of the microorganisms (P. aeruginosa, characterized by the yellow-green pigment on the PIA plates; S. aureus, characterized by the orange-magenta color on the C-agar plates). Unconcentrated (unfiltered) control samples had 140 CFUs/mL of P. aeruginosa and 170 CFUs/mL of S. aureus, respectively. Results are shown in Table 6.

TABLE 6

| | Concentration | | Microorganism Capture |
|---|---|---|---|
| Example No. | Device | Microorganism | Efficiency (%) |
| 11 | 1 | P. aeruginosa | 78 |
| 12 | 1 | S. aureus | 96 |
| 13 | 2 | P. aeruginosa | 99 |
| 14 | 2 | S. aureus | 99 |

### Examples 15-16

### Water Filtration

A streak culture of E. coli (ATCC 51813) was prepared on a Blood Agar plate (Tryptic Soy Agar with 5% sheep's blood; Hardy Diagnostics; Santa Maria, Calif.) and incubated at 37° C. overnight. The culture was used to prepare a 0.5 McFarland Standard using DensiCHEK™ densitometer (bioMerieux, Inc., Durham, N.C.) in 3 mL Butterfield's Buffer. The resulting bacterial stock, containing 10⁸ cfus/mL, was serially diluted in Butterfield's to obtain an inoculum having approximately 10⁶ cfus/mL.

A test sample was prepared by inoculating 100 mL deionized of water (MilliQ Gradient system, Millipore, Ma) a 1:100 dilution of the 10⁶ bacteria/ml inoculum resulting in water test sample containing 10⁴ CFU/ml (10⁶ CFUs total in the water).

The inoculated water sample was pumped through a filtration device holding a 47 mm diameter die cut disk of the fibrous nonwoven matrix shown in Table 7. The device had a polycarbonate cylindrical body measuring about 60 mm in diameter and about 115 mm high and having a support screen to hold the filter disk in the body. The top end of the body was closed with a threaded cap having an inlet port attached to a peristaltic pump (Model No. 7553-70; Cole Parmer) by ⅛″ thick wall PVC tubing (Catalog #60985-522; VWR; Batavia, Ill.). The pump was used to deliver the water sample to the filtration device. The bottom end of the cylinder was closed with a threaded section having an outlet port. O-rings were positioned between the threaded parts to prevent leakage. The device was vented on the upstream side to allow for purging of air.

The result for Example 15 was based on a single test and the result for Example 16 was the average of 2 duplicates. For each test, a 100 mL sample of inoculated water was pumped into the filtration device at a flow rate of 70 ml/minute. Filtrates were collected in sterile 100 ml polypropylene beakers. After each filtration test, the device was disassembled and the disk was removed using sterile forceps. Between each test, the filtration device was rinsed with 500 mL of filtered sterilized deionized water.

One hundred microliter volumes of each filtrate and a pre-filtration suspension, were diluted 1:10 and 1:100 in Butterfield's Buffer and plated onto AC Plates. The plates were incubated at 37° C. for 18-20 hours. Colony counts were determined from the plates according to the manufacturer's instructions. The Log Reduction Value (LRV) is an indication of the bacterial removal capacity of a water filter. The values were calculated based on counts obtained from the plated filtrate and pre-filtration samples by using the formula below:

LRV=(Log of CFUs/ml in pre-filtration sample)−(Log of CFUs/ml in filtrate sample)

The pre-filtration suspension contained an average of 8500 CFU/ml (3.9 Log CFU/ml).

Results are shown in Table 7.

TABLE 7

| Example | Disk | LRV |
|---|---|---|
| 15 | Example 1 | 3.9 |
| 16 | Example 2 | 3.9 |

The referenced descriptions contained in the patents, patent documents, and publications cited herein are incorporated by reference in their entirety as if each were individually incorporated. Various unforeseeable modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only, with the scope of the invention intended to be limited only by the claims set forth herein as follows:

1. A concentration process comprising
(a) providing a concentration device comprising
(1) a porous fibrous nonwoven matrix, and
(2) a plurality of particles of at least one concentration agent that comprises diatomaceous earth, said particles being enmeshed in said porous fibrous nonwoven matrix;
(b) providing a sample comprising at least one target cellular analyte;
(c) contacting said concentration device with said sample such that at least a portion of said at least one target cellular analyte is bound to or captured by said concentration device; and
(d) detecting the presence of at least one bound target cellular analyte.

2. The process of claim 1, wherein said porous fibrous nonwoven matrix is formed by a wetlaid process.

3. The process of claim 1, wherein said porous fibrous nonwoven matrix comprises at least one fibrillated fiber.

4. The process of claim 1, wherein the fibers of said porous fibrous nonwoven matrix are selected from polymer fibers, inorganic fibers, and combinations thereof.

5. The process of claim 4, wherein said polymer fibers comprise at least one polymer selected from polyamides, polyolefins, polysulfones, and combinations thereof.

6. The process of claim 4, wherein said inorganic fibers comprise at least one inorganic material selected from glasses, ceramics, and combinations thereof.

7. The process of claim 1, wherein said porous fibrous nonwoven matrix comprises at least one polymeric binder.

8. The process of claim 7, wherein said polymeric binder is selected from polymeric resins, polymeric binder fibers, and combinations thereof.

9. The process of claim 7, wherein said polymeric binder does not substantially adhere to said particles of concentration agent.

10. The process of claim 1, wherein said particles are mechanically entrapped in said porous fibrous nonwoven matrix.

11. The process of claim 1, wherein said particles comprise microparticles.

12. The process of claim 1, wherein said diatomaceous earth has been surface-modified to enhance its ability to concentrate microorganisms.

13. The process of claim 1, wherein said diatomaceous earth bears, on at least a portion of its surface, a surface treatment comprising a surface modifier comprising metal oxide, fine-nanoscale gold or platinum, or a combination thereof.

14. The process of claim 1, wherein said diatomaceous earth bears, on at least a portion of its surface, a surface treatment comprising a surface modifier comprising at least one metal oxide.

15. The process of claim 1, wherein said diatomaceous earth bears, on at least a portion of its surface, a surface treatment comprising a surface modifier comprising at least one metal oxide selected from titanium dioxide, ferric oxide, and combinations thereof.

16. The process of claim 1, wherein said sample is in the form of a fluid.

17. The process of claim 1, wherein said target cellular analyte is selected from cells of bacteria, fungi, yeasts, protozoans, viruses, bacterial endospores, components thereof, and combinations thereof.

18-21. (canceled)

22. A concentration device comprising
(a) a porous fibrous nonwoven matrix; and
(b) a plurality of particles of at least one concentration agent that comprises diatomaceous earth bearing, on at least a portion of its surface, a surface treatment comprising a surface modifier comprising metal oxide, fine-nanoscale gold or platinum, or a combination thereof;
wherein said particles are enmeshed in said porous fibrous nonwoven matrix.

23. A kit comprising
(a) at least one concentration device of claim 22; and
(b) at least one testing container or testing reagent for use in carrying out the process of claim 1.

24. A process for preparing a concentration device comprising
(a) providing a plurality of fibers;
(b) providing a plurality of particles of at least one concentration agent that comprises diatomaceous earth bearing, on at least a portion of its surface, a surface treatment comprising a surface modifier comprising metal oxide, fine-nanoscale gold or platinum, or a combination thereof; and
(c) forming at least a portion of said plurality of fibers into a porous fibrous nonwoven matrix having at least a portion of said plurality of particles enmeshed therein.

25-27. (canceled)
